(19) United States
(12) Patent Application Publication
(10) Pub. No.: US 2026/0151362 A1
(43) Pub. Date: Jun. 4, 2026

(54) A pharmaceutical composition for effectively preventing deactivation and enhancing the targeting activity of SN triple-bond compounds, and a method for preparing the same

(71) Applicant: SHANGHAI JZIY BIOTECHNOLOGY CO., LTD., Shanghai (CN)

(72) Inventors: Wei HAO, Shanghai (CN); Yangfan HAO, Shanghai (CN); Yali QIAO, Shanghai (CN)

(73) Assignee: SHANGHAI JZIY BIOTECHNOLOGY CO., LTD., Shanghai (CN)

(21) Appl. No.: 18/704,948

(22) PCT Filed: Oct. 18, 2022
(86) PCT No.: PCT/CN2022/125824

§ 371 Date: Apr. 26, 2024

(30) Foreign Application Priority Data
Nov. 1, 2021 (CN) 202111284606.9

(51) Int. Cl. A61K 31/145; A61K 47/02; A61K 47/10; A61K 47/36; A61K 47/38

(52) U.S. Cl. CPC A61K 31/145; A61K 47/02; A61K 47/10; A61K 47/36; A61K 47/38

(57) ABSTRACT

The pharmaceutical composition capable of effectively preventing deactivation and enhancing targeting activity of SN triple-bond structured compound, comprises an SN triple-bond structured compound, a stabilizer protectant, and a pharmaceutical excipient. The pharmaceutical composition of the present disclosure can ensure that the SN triple-bond structured compound is located in the environment lacking proton and water and being hydrophobic, improve its targeting ability and acid avoidance properties, and avoid its reaction with other easy-to-react reactants, thus laying the foundation for applying the pharmaceutical composition to medications for resisting tumors and the like.

### FIELD OF TECHNOLOGY

The present disclosure relates to the field of pharmaceutical technology, and particularly to a pharmaceutical composition capable of effectively preventing deactivation and enhancing targeting activity of SN triple-bond structured compound, and preparation method thereof.

### BACKGROUND

SN triple-bond structured compounds are a class of synthetic compounds with sulfur-nitrogen triple bond. SN triple-bond structured compounds are a rare class of compounds with a functional group of SEN and are lipophilic small-molecule compounds, whose sulfur-nitrogen triple bond is very reactive and can be easily protonated, and which have a very strong proton-capturing activity from piperidine analogues. Therefore, in the proton-containing site of chromosomes in cells, whose structure is similar to the piperidine, the SN triple-bond structured compounds can block the process of DNA, RNA, and protein synthesis by seizing protons, which makes it difficult for cancer cells to divide and proliferate, and very promising in terms of application. At the same time, if these compounds contain an alkoxy group attached to the sulfur atom, due to the protonation of the sulfur-nitrogen bond the compounds will contain a highly active alkylation group, and can bind to the nucleic acids of body cells to inhibit and destroy cancer cells, etc., thereby enhancing the autoimmune effects. The most important feature of these compounds is that under certain conditions, the sulfur-nitrogen triple bond is relatively easy to seize the protons on the piperidine analogues, causing them to lose their replication ability, which further inserts the alkyl group into the base chain, accelerating the blocking of base replication, protein synthesis and so on. At the same time, the sulfur-nitrogen triple bond is particularly easy to protonate in acidic medium, and its alkylation activity is particularly high, which can easily alkylate the base chain and block cell proliferation. The biggest limitation of such compounds is that in an acidic environment, the sulfur-nitrogen triple bond functional group is easy to be destroyed in acidic medium, causing the compounds unable to protonate, and then unable to seize protons from DNA, RNA and other base to block their replication and proliferation. After protonation, the sulfur-nitrogen triple bond compounds are quickly unable to block the base chain. And in the solvent SN triple bond compounds are also very difficult to react with piperidine to seize the proton on its molecule, which will greatly weaken the anti-cancer and anti-bacterial effect and other practical utility, therefore this class of compounds has not been applied as pharmaceuticals. How to effectively protect the activity of such compounds before they encountering the target, that is, when reaching the target, how to make the compounds very active and can efficiently seize protons from piperidine analogues, and have a long residence time and strong targeting are still challenging.

Furthermore, the SN-triple structured bond compounds are susceptible to hydrolyze and deactivate. It is well known that many pharmaceuticals are easy to hydrolyze or deliquesce, so that their efficacy are greatly weakened or even ineffective. To avoid hydrolysis, they can be made into lyophilized powder, injection, injection powder, tablets, capsules, granules according to the characteristics of different drugs, and the route of administration comprises intravenous drug delivery, oral and perfusion. Additionally, to minimize the efficacy loss, it also requires a scientific method to ensure the stability of the active pharmaceuticals, which will be an important issue that needs to be further addressed for specific clinical applications.

Currently, due to the SN-triple structured bond compounds are easy to hydrolyze and deactivate, there have been no reports of their use as inhibitors of cell reproduction and anti-tumor drugs. Therefore, it is necessary to develop a formulation of SN triple bond compounds that are not susceptible to deliquescence, hydrolysis, decomposition, and has a high activity against the target, which not only prevents the hydrolysis and decomposition of SN triple bond drugs in the industrial production process, but also maintains the stability and high targeting activity of the active drug before reaching the target cell, and reduces the decomposition of SN triple bond compounds.

### SUMMARY

The present disclosure provides a pharmaceutical composition capable of effectively preventing deactivation and enhancing targeting activity of SN triple-bond structured compound and preparation method thereof, which ensures that the SN triple bond compound is located in the environment lacking of proton and water and being hydrophobic, has the targeting activity and acid avoidance properties, and avoids reaction with other easy-to-react reactants, thereby laying the foundation for applying the pharmaceutical composition to medications for resisting tumors and the like in the future.

To achieve the above object and other related purposes, the present disclosure adopts the following technical solutions:

The present disclosure provides a pharmaceutical composition, the pharmaceutical composition includes, by mass percent:

SN triple-bond structured compound 2%˜98%;
stabilizer protectant 0.5%˜97%;
pharmaceutical excipient 0.1%˜55%.

In some embodiments of the present disclosure, the pharmaceutical composition further includes a lubricant. The pharmaceutical composition includes, by mass percent:
SN triple-bond structured compound 40%˜85%;
stabilizer protectant 0.5%˜8%;
pharmaceutical excipient 5%˜55%;
lubricant 2%˜18%.

In some embodiments of the present disclosure, the pharmaceutical composition is dissolved in a solvent when used. Preferably, the solvent is anhydrous ethanol.

In some embodiments of the present disclosure, the SN triple-bond structured compound is selected from APSNRI and FPSNRI.

In some embodiments of the present disclosure, the stabilizer protectant is one or more of the following components: sodium alcoholates, alcohols, alcohol polymers, pyridines, proton-deficient hydrophobic oils and the like. The stabilizer protectant provides SN triple-bond structured compounds with an environment where lacks active protons and enhances their targeting activity. Preferably, the stabilizer protectant is one or more of the following components: sodium ethoxide, 4-dimethylaminopyridine, polyethylene glycol monoalkyl ether, and anhydrous ethanol.

In some embodiments of the present disclosure, the pharmaceutical excipient is one or more of the following components: microcrystalline cellulose, pregelatinized starch, and capsules.

In some embodiments of the present disclosure, the lubricant is one or more of the following components: talc powder, magnesium stearate, and microcrystalline silica. Preferably, the lubricant is microcrystalline silica.

The present disclosure provides a method for preparing the pharmaceutical composition, including mixing the SN triple-bond structured compound, the stabilizer protectant, and the pharmaceutical excipient.

In some embodiments of the present disclosure, the method further includes adding the lubricant to the mixture of the SN triple-bond structured compound, the stabilizer protectant, and the pharmaceutical excipient.

The present disclosure provides a use of the pharmaceutical composition in drugs for inhibiting cell reproduction anti-tumor.

### DETAILED DESCRIPTION

Hereinafter, the detailed description specifies the embodiments of the pharmaceutical composition capable of effectively preventing deactivation and enhancing targeting activity of SN triple-bond structured compound.

The “range” disclosed herein is represented in the form of a lower limit and an upper limit, and the given range is defined by selecting a lower limit and an upper limit, which set the boundaries of the particular range. The ranges that are limited in this manner may be inclusive of end values or exclusive of end values and may be combined in any way, i.e. any lower limit may be combined with any upper limit to form a range. For example, if ranges of 60-120 and 80-110 are listed for a particular parameter, it should be understood that ranges of 60-110 and 80-120 are also expected. In addition, if the minimum lower limits 1 and 2 are listed, and if the maximum upper limits 3, 4, and 5 are listed, the following ranges can all be expected: 1-3, 1-4, 1-5, 2-3, 2-4, and 2-5. In the present disclosure, unless otherwise indicated, the range “a-b” is an abbreviated representation of any ranges which combine any real numbers between a and b, where a and b are also real numbers. For example, the range “0-5” of the present disclosure has listed all real numbers in “0-5”, and “0-5” is just an abbreviated representation of the combination of these real numbers. Further, when expressing that a parameter is an integer ≥2, it should be understood that the parameter is, for example, an integer 2, 3, 4, 5, 6, 7, 8, 9, 10, 11, 12, etc.

If not specifically stated, all embodiments of the present disclosure, as well as optional embodiments, can be combined with each other to form new technical solutions.

If not specifically stated, all technical features of the present disclosure as well as optional technical features can be combined with each other to form new technical solutions.

If not specifically stated, all steps of the present disclosure may be performed sequentially or randomly, preferably sequentially. For example, the method includes steps (1) and (2), indicating that the method may comprise steps (1) and (2) performed sequentially or may comprise steps (2) and (1) performed sequentially. For example, the method may further comprise step (3), which may be added to the method and performed in any order, i.e., the method may comprise steps (1), (2), and (3), or may comprise steps (1), (3), and (2), or may comprise steps (3), (2), and (1), and so on.

Unless otherwise specified, to the terms “include” and “comprise” in this application are open-ended and may be closed-ended. For example, the words “include” and “comprise” may indicate that other components not listed may also be included or comprised, or that only the listed components may be included or comprised.

In this application, the term “or” is inclusive if not otherwise indicated. For example, the phrase “A or B” means “A, B, or both A and B”. More specifically, either of the following satisfies the condition “A or B”: A is true (or exists) and B is false (or does not exist); A is false (or does not exist) and B is true (or exists); or both A and B are true (or exist).

The applicant has carried out a lot of research on the deprotonation and alkylation of sulfur-nitrogen triple bond compounds in piperidine analogues, the results showed that sulfur-nitrogen triple bond compounds had very high activity below 40 degrees, and their biggest shortcoming is that they are easy to decompose and deactivate in acidic medium, and to hydrolyze and decompose, thus losing the deprotonation and alkylation ability.

After much exploration and experimentation, the present disclosure provides a pharmaceutical composition and preparation thereof, the pharmaceutical composition is capable of effectively preventing SN triple-bond structured compounds from being hydrolyzed and decomposed, and enhancing targeting activity, deprotonation activity, and alkylating activity of SN triple-bond structured compounds. The pharmaceutical composition also ensures that the SN triple bond compound is located in the environment lacking of proton and water and being hydrophobic, has the targeting activity and acid avoidance properties, and avoids reaction with other easy-to-react reactants, thereby laying the foundation for applying the pharmaceutical composition to medications for resisting tumors and the like.

The first aspect of the present disclosure provides a pharmaceutical composition for providing the SN triple bond compound susceptible to hydrolysis and decomposition with an environment that lacks active protons and water, as well as enabling the SN triple bond compound to effectively seize protons from piperidinium analogues, and effectively enhancing the targeting activity. The pharmaceutical composition includes an active ingredient (a SN triple-bond structured compound), a stabilizer protectant, and a pharmaceutical excipient. The stabilizer protectant can make the pharmaceutical composition provides the SN triple bond compound with an environment lacking protons and water and being hydrophobic, thus enhancing the deprotonation activity and keeping the compound in a hydrophobic state, so that the SN triple-bond structured compound can be effectively prevented from being deactivated and the targeting activity can be improved.

The pharmaceutical composition of the present disclosure typically contains a certain percentage of SN triple-bond structured compounds. The pharmaceutical composition includes 2% to 98% of the SN triple-bond structured compound in terms of mass percentage. In some embodiments, the mass percentage of the SN triple-bond structured compound may also be in a range of 2%˜40%, 40%˜85%, 40%˜85%, 85%˜98%, 40%˜50%, 50%˜80%, 80%˜85%, 40%˜60%, 60%˜80%, 5%˜90%, 10%˜80%, 20%˜70%, 30%˜60%, or 40%˜70%, etc.

The molecular formula of SN triple bond compounds can be represented by the following:

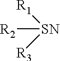

R₁, R₃ are arylphenyl; R₂ is OCH₃, OC₂H₅, OCH₇, F or the like.

In some embodiments, the SN triple-bond structured compound may, for example, be selected from APSNRI and FPSNRI. For ease of use, when R₂ is OCH₃, OC₂H₅, OCH₇, etc., and R₁ and R₅ are arylphenyl, the SN triple bond compound is usually referred to as APSNRI (Alkoxy(aryl)(phenyl)-λ⁶-sulfanenitriles); when R₂ is F, and R₁ and R₃ are arylphenyl, the SN triple bond compound is referred to as FPSNRI (Aryl(fluoro)(phenyl)-λ⁶-sulfanenitriles). Arylphenyl includes phenyl and phenyl with substituents. The substituent may, for example, be an alkyl group, a halogen group, a nitro group, and the like. APSNRI has a molecular weight greater than or equal to 231. In a specific embodiment, when R₂ is OCH₃, and R₁ and R₃ are both phenyl, the molecular weight of APSNRI is 231. The molecular weight of FPSNRI is greater than or equal to 219. In a specific embodiment, when R₂ is F, and R₁ and R₃ are both phenyl, the molecular weight of FPSNRI is 219.

The pharmaceutical composition of the present disclosure typically also contain a certain percentage of the stabilizer protectant. The stabilizer protectant herein further serves as a protective agent, and in some embodiments, the stabilizer protectant can also serve as an activating agent for enhancing the targeting activity of the SN triple-bond structured compound. The pharmaceutical composition includes 0.5% to 97% of the stabilizer protectant in terms of mass percentage. Specifically, in some embodiments, the mass percentage of the stabilizer protectant may also be in a range of 0.5%˜8%, 8%˜20%, 20%˜40%, 40%˜60%, 60%˜80%, 80%˜97%, 0.5%˜1%, 1%˜6%, 6%˜8%, 1%˜5%, 5%˜8%, 1%˜95%, 5%˜90%, 10%˜85%, 20%˜75%, 30%˜65%, or 40%˜55%, etc.

The compounds that can provide the SN triple-bond structured compound with the ability to accept protons and effectively enhance targeting activity can be selected as a stabilizer protectant of the pharmaceutical composition of the present disclosure. In some embodiments of the present disclosure, the stabilizer protectant is one or more of the following components: sodium alcoholates, alcohols, alcohol polymers, pyridines, proton-deficient hydrophobic oils. Preferably, the stabilizer protectant is one or more of the following components: sodium ethoxide, 4-dimethylaminopyridine, polyethylene glycol monoalkyl ether, anhydrous ethanol. The molecular formula of the polyethylene glycol monoalkyl ether is HO(CH₂CH₂O)ₙC₁₂H₂₅, and the molecular weight can be, for example, 1100 to 1300, 1100 to 1200, 1200 to 1300, and the like. More preferably, the stabilizer protectant is sodium alcoholates and/or polyethylene glycol monoalkyl ether. Alternatively, in some embodiments, the stabilizer protectant contains other substances, such as sodium chloride, in addition to the alcohol group lacking protons.

The reason for employing sodium alcoholates, DMAP, or polyethylene glycol monoalkyl ether, etc., which lack protons and can accept protons, as the stabilizer protectant for the SN triple-bond structured compound, is that the hydrolysis of the SN triple-bond structured compound is a first-order reaction as indicated by the pH-rate distributions and the hydrolysis under acidic conditions is relatively fast, e.g., at 25 degrees, with the solvent of D₂O/CD₃CN (phosphate buffer 8:1), pH of 6.1, and a total buffer concentration of 0.065 mol-dm 3, 99% of the ethoxy SN triple-bond structured compound (0.035 mol-dm 3) were hydrolyzed within 45 min.

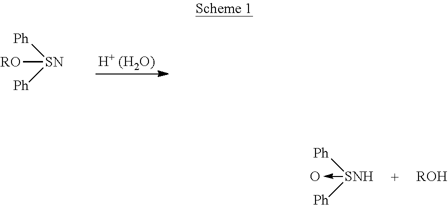

In addition, if an appropriate amount of a compound lacking protons and capable of accepting protons and hydrogen ions or an appropriate amount of a deprotonated compound, such as sodium alcoholates, DMAP, polyethylene glycol monoalkyl ether, etc., is provided in the pharmaceutical composition, on the one hand, it can keep the partial environment weakly alkaline, which is useful for preventing hydrolysis of the SN triple-bond structured compound; on the other hand, the number of protons in the environment is greatly decreased, reducing the protonation of the SN triple bond and thus reducing the decomposition and deactivation of the SN triple bond. For the present disclosure, the preferred stabilizer protectant is firstly that may reduce the hydrogen ion concentration in the environment; secondly that may reduce the water content in the environment, which also absorbs a small amount of water in the environment; thirdly that may allow the pharmaceutical composition to be hydrophobic and easily soluble in piperidine analogues.

The pharmaceutical composition of the present disclosure typically also contains a certain percentage of the pharmaceutical excipient. The pharmaceutical composition includes 0.1% to 55% of the pharmaceutical excipient in terms of mass percentage. In some embodiments, the mass percentage of the pharmaceutical excipient may also be in a range of 0.1% to 5%, 5% to 55%, 5% to 10%, 10% to 45%, 45% to 55%, 10% to 35%, 35% to 55%, 1% to 50%, 5% to 45%, 10% to 40%, 20% to 35%, or the like.

In the pharmaceutical composition of the present disclosure, the pharmaceutical excipient with a low hygroscopicity is selected to further ensure the stability of the pharmaceuticals during storage and use. Lactose is a common excipient used in pharmaceuticals, but its high hygroscopicity is detrimental to the stability of the SN triple-bond structured compound when performing tabletting, as well as to the storage of the prepared tablets. The pregelatinized starch has a certain degree of hygroscopicity, which is detrimental to the stability of the SN triple-bond structured compound when performing tabletting, but the pregelatinized starch has good compressibility and fluidity, when it is used in conjunction with microcrystalline cellulose, more optimal effects can be achieved. Therefore, in the pharmaceutical composition of the present disclosure, the pharmaceutical excipient is one or more of the following components: microcrystalline cellulose, pregelatinized starch, and capsules. Preferably, the pharmaceutical excipient is microcrystalline cellulose and/or pregelatinized starch, wherein the microcrystalline cellulose has the molecular formula of (C₆H₁₀O₅)_{n/2} and the degree of polymerization of n≤350. Microcrystalline cellulose ph103 and pregelatinized starch are used in the experiments of the present disclosure; wherein the pregelatinized starch has the molecular formula of (C₆H₁₀O₅)ₙ and degree of polymerization of n=300 to 1000.

A more preferred pharmaceutical excipient is a combination of microcrystalline cellulose and pregelatinized starch. The mass ratio of microcrystalline cellulose to pregelatinized starch is 1˜2:1˜4, 1˜2:1˜2, 1˜2:2˜3, 1˜2:3˜4, etc.

In a specific embodiment, the pharmaceutical composition includes, by mass percent:
SN triple-bond structured compound 2%˜98%;
stabilizer protectant 0.5%˜97%;
pharmaceutical excipient 0.1% to 55%.

For the pharmaceutical composition of the present disclosure, typically, different pharmaceutical excipients can be selected corresponding to the targeting site. If the targeting site is in the stomach, intestine, esophagus, etc. lubricants may be required. Thus, the pharmaceutical composition may further comprise a lubricant.

In the pharmaceutical composition of the present disclosure, the pharmaceutical composition includes 2% to 18% of the lubricant in terms of mass percentage. The mass percentage of the lubricant may also be in a range of 2% to 3%, 3% to 15%, 15% to 18%, 2% to 5%, 5% to 10%, 10% to 18%, 3% to 15%, 5% to 12%, 8% to 10%, or the like.

In the pharmaceutical composition of the present disclosure, the lubricant is one or more of the following components: talc powder, magnesium stearate, and microcrystalline silica. Because of the high water absorption capacity of microcrystalline silica, it is usually used as a desiccant for chemicals and medical products to prevent moisture. Microcrystalline silica can be used as a purifying agent for partial water in tablets, which is used to absorb free water in the micropores of tablets to reduce the interaction between water and pharmaceuticals molecules, and also has a certain stabilizing effect for the SN triple-bond structured compound. Magnesium stearate may contain free metal ions, which catalyze the hydrolysis of SN triple-bond structured compound. Therefore, microcrystalline silica is preferable, which not only provides the lubricating effect but also the stabilizing protective effect.

In a specific embodiment, the pharmaceutical composition includes, by mass percent:
SN triple-bond structured compound 40%˜85%;
stabilizer protectant 0.5%˜8%;
pharmaceutical excipient 5%˜55%;
lubricant 2%˜18%.

In a preferred specific embodiment, the pharmaceutical composition includes, by mass percent:
SN triple-bond structured compound 50%˜80%;
stabilizing protectant 1%˜6%;
pharmaceutical excipient 10%˜45%;
lubricant 3%˜15%.

In a more preferred specific embodiment, the pharmaceutical composition includes, by mass percent:
SN triple-bond structured compound 60%˜80%;
stabilizing protectant 1%˜5%;
pharmaceutical excipient 10%˜35%;
lubricant 5%˜10%.

The pharmaceutical composition of the present disclosure can be dissolved in a solvent. Preferably, the solvent is anhydrous ethanol. Anhydrous ethanol is used in an amount sufficient to dissolve the pharmaceutical composition, for example, a mass ratio of the pharmaceutical composition to the anhydrous ethanol is 1˜8:5˜30. In some embodiments, the mass ratio of the pharmaceutical composition to the anhydrous ethanol may also be in a range of 1˜3:5˜30, 3˜5:5˜30, 5˜8:5˜30, 1˜8:5˜10, 1˜8:10˜20, 1˜8:20˜30, or the like.

Anhydrous ethanol can directly dissolve the SN triple-bond structured compound, stabilizer protectant, pharmaceutical excipient, etc. in the pharmaceutical composition, and the mixture is then directly injected into the solution containing piperidine (simulating the biological environment). The pharmaceutical composition makes it possible to effectively protect the activity of such SN triple-bond structured compounds before reaching the targeting site, even when near the targeting site, the environmental state lacking active protons or hydrogen ions, lacking water, and being hydrophobic are realized for the SN triple-bond structured compound. The pharmaceutical composition may reach piperidine analogues easily and then is soluble in piperidine analogues, is very reactive and efficiently able to seize protons from piperidine analogues, and has a long residence time and high targeting ability.

A second aspect of the present disclosure provides a method for preparing the pharmaceutical composition described in the first aspect of the present disclosure, including mixing the SN triple-bond structured compound, the stabilizer protectant, and the pharmaceutical excipient.

If used in the stomach, intestine, esophagus, etc. the pharmaceutical composition may further include the lubricant. In an embodiment of the present disclosure, the method further includes adding the lubricant to the mixture of the SN triple-bond structured compound, the stabilizer protectant, and the pharmaceutical excipient.

In the method for preparing the pharmaceutical composition of the present disclosure, the pharmaceutical composition can be made in the form of tablets, capsules and other preparations. If the direct compression method is performed on all the powder, the drying process of wet granulation can be eliminated, thus reducing the possibility of hydrolysis of the SN triple-bond structured compound during the production process.

In addition, in terms of the packaging of the preparation, the packaging material is also an important part for improving the stability of the drug. A layer of aluminum foil may wrap on the outside of the drug and the drug may be sealed individually to better isolate itself from the air and prevent hydrolysis. Experiments have shown that the stability of the SN triple-bond structured compound was greatly improved by employing aluminum foil.

The present disclosure provides a use of the pharmaceutical composition in drugs for inhibiting cell reproduction and anti-tumor. When the pharmaceutical composition is used in drugs for inhibiting cell reproduction and anti-tumor, the stability of the active drug before reaching the target cell and high targeting activity are maintained, so as to reduce the decomposition of SN triple-bond structured compound.

The pharmaceutical composition and preparation thereof of the present disclosure can effectively prevent the hydrolysis of the SN triple-bond structured compound, in which the stabilizer protectant can enable the pharmaceutical composition to be located in the environment lacking active protons and water and being hydrophobic, and can enhance the targeting activity of the compound, effectively enhancing the deprotonation activity and alkylation activity to the targeting site. The pharmaceutical composition skillfully applies the timing gap of ‘like dissolve like’, so that the deprotonation activity can be greatly enhanced over time, which not only extends the active time of the pharmaceuticals but also enhances the deprotonation and alkylation activity. Therefore, a solid foundation has been laid for subsequent clinical trials.

The implementation of the present disclosure will be described below through exemplary embodiments. Those skilled in the art can easily understand other advantages and effects of the present disclosure according to contents disclosed by the specification. The present disclosure may also be implemented or applied through other different specific embodiments. Various modifications or changes may be made to all details in the specification based on different points of view and applications without departing from the spirit of the present disclosure.

In the following embodiments, the reagents, materials, and instruments used are commercially available if not otherwise specified.

The experimental materials used in the following experiments include: APSNRI, FPSNRI, sodium ethoxide, DMAP, polyethylene glycol monoalkyl ether, microcrystalline silica, microcrystalline cellulose, pregelatinized starch, and anhydrous ethanol. The equipment used mainly includes the tablet press machine (19 tablet press), electro-heating constant temperature incubator, etc. Tablet and solvent: the tablet is dissolved in anhydrous ethanol and then injected into the piperidine part when using; the solvent is anhydrous ethanol which works directly by injecting it into the piperidine part when using. The stability, deprotonation activity and other efficacy profiles of the pharmaceutical composition containing the SN triple-bond structured compound of the present disclosure are analyzed and determined by the change in the area ratio of the nuclear magnetic resonance spectroscopy ¹H NMR, which are commonly used methods. The reaction products of piperidine in the present disclosure can be analyzed against the reaction products synthesized by other known methods or commercially purchased counterparts, such as the reaction products of Scheme 2.

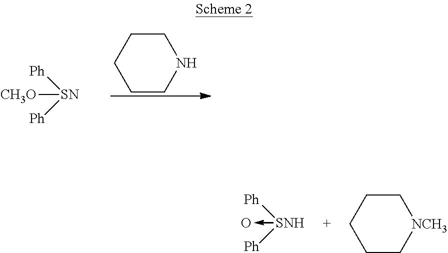

APSNRI is synthesized from the reaction of FPSNRI, and raw materials were purchased from Sinopharm Chemical Reagent Co., Ltd. Synthesis and Preparation may refer to: 1) Bull. Chem. Soc. Jpn. 71, 1629-1637 (1998), Yoshimura, T; Ohkubo, M.; Fujii, T.; Kita, H.; Wakai, Y.; Ono, S.; etc.

FPSNRI is synthesized from a multi-step reaction of phenyl sulfide and chloramine T. The raw materials were purchased from Sinopharm Chemical Reagent Co., Ltd. Synthesis and preparation may refer to: 1) Tetrahedron Lett, 30, 6339-6340(1989), Yoshimura, T; Tsukurimichi, E.; Kita, H.; Fujii, H.; Shimasaki, C.; 2) a. Chem. Lett. 1433-1436(1992), Yoshimura, T, etc.; b. Chem. Lett. 2213-2216(1992), Yoshimura, T, etc.; c. J. Org. Chem. 62. 3802-3803(1997), Yoshimura, T, etc.

The microcrystalline silica (with 200 to 300 mesh) was purchased from Lianshuo Biotechnology Co., Ltd.

The microcrystalline cellulose was purchased from Snow Season Food Ingredients Company and produced in accordance with GB1886.103 by Huzhou Linghu Xinwang Chemical Co., Ltd.

The pregelatinized starch was purchased from Shanghai Qianwei Food Additives Company.

The polyethylene glycol monoalkyl ether was purchased from the Research Reagents and Supplies Store (Brij-35).

### Comparative Example 1

For preparing the pharmaceutical composition containing 100 mg of the active ingredient APSNRI per tablet, the formulation is as follows:
APSNRI 100 mg

### Detailed Preparing Method

The SN triple-bond structured compound, employing direct compression method, was mixed uniformly and pressed to produce a tablet containing 100 mg of the SN triple-bond structured compound.

### Comparative Example 2

For preparing the pharmaceutical composition containing 100 mg of the active ingredient APSNRI per tablet, the formulation is as follows:
APSNRI 100 mg
sodium ethoxide 5 mg
DMAP 20 mg
polyethylene glycol monoalkyl ether 10 mg

### Detailed Preparing Method

The SN triple-bond structured compound, sodium ethoxide, DMAP, and polyethylene glycol monoalkyl ether, employing direct compression method, were mixed uniformly and pressed to produce a tablet containing 100 mg of the SN triple-bond structured compound.

### Comparative Example 3

For preparing the pharmaceutical composition containing 100 mg of the active ingredient APSNRI per tablet, the formulation is as follows:
APSNRI 100 mg
DMAP 20 mg
polyethylene glycol monoalkyl ether 10 mg

### Detailed Preparing Method

The SN triple-bond structured compound, DMAP, and polyethylene glycol monoalkyl ether, employing direct compression method, were mixed uniformly and pressed to produce a tablet containing 100 mg of the SN triple-bond structured compound.

### Comparative Example 4

For preparing the pharmaceutical composition containing 100 mg of the active ingredient APSNRI per tablet, the formulation is as follows:
APSNRI 100 mg
polyethylene glycol monoalkyl ether 10 mg

### Detailed Preparing Method

The SN triple-bond compound and the polyethylene glycol monoalkyl ether, employing direct compression method, were mixed uniformly and pressed to produce a tablet containing 100 mg of the SN triple-bond structured compound.

### Comparative Example 5

For preparing the pharmaceutical composition containing 100 mg of the active ingredient APSNRI per tablet, the formulation is as follows:
APSNRI 100 mg
DMAP 20 mg

### Detailed Preparing Method

The SN triple-bond structured compound and DMAP, employing direct compression method, were mixed uniformly and pressed to produce a tablet containing 100 mg of the SN triple-bond structured compound.

### Comparative Example 6

For preparing the pharmaceutical composition containing 100 mg of the active ingredient APSNRI per tablet, the formulation is as follows:
APSNRI 100 mg
sodium ethoxide 5 mg

### Detailed Preparing Method

The SN triple-bond structured compound and the sodium ethoxide, employing direct compression method, were mixed uniformly and pressed to produce a tablet containing 100 mg of the SN triple-bond structured compound.

### Embodiment 1

According to Comparative Example 1, the same method for preparing the pharmaceutical composition containing 100 mg of the active ingredient APSNRI per tablet was employed, the formulation is as follows:
APSNRI 100 mg
sodium ethoxide 8 mg
DMAP 10 mg
polyethylene glycol monoalkyl ether 20 mg
microcrystalline cellulose 20 mg;
pregelatinized starch 40 mg

### Detailed Preparing Method

Tablet preparation: the SN triple-bond structured compound, sodium ethoxide, DMAP, polyethylene glycol monoalkyl ether, microcrystalline cellulose, and pregelatinized starch, employing direct compression method, were mixed uniformly and pressed to produce a tablet containing 100 mg of the SN triple-bond structured compound.

### Embodiment 2

According to Comparative Example 1, the same method for preparing the pharmaceutical composition containing 100 mg of the active ingredient APSNRI per tablet was employed, the formulation is as follows:
APSNRI 100 mg
sodium ethoxide 5 mg
DMAP 5 mg
polyethylene glycol monoalkyl ether 20 mg
microcrystalline cellulose 20 mg

### Detailed Preparing Method

Tablet preparation: the SN triple-bond structured compound, sodium ethoxide, DMAP, polyethylene glycol monoalkyl ether, and microcrystalline cellulose, employing direct compression method, were mixed uniformly and pressed to produce a tablet containing 100 mg of the SN triple-bond structured compound.

### Embodiment 3

According to Comparative Example 1, the same method for preparing the pharmaceutical composition containing 100 mg of the active ingredient APSNRI per tablet was employed, the formulation is as follows:
APSNRI 100 mg
sodium ethoxide 5 mg
DMAP 5 mg
polyethylene glycol monoalkyl ether 20 mg
pregelatinized starch 40 mg

### Detailed Preparing Method

Tablet preparation: the SN triple-bond structured compound, sodium ethoxide, DMAP, polyethylene glycol monoalkyl ether, and pregelatinized starch, employing direct compression method, were mixed uniformly and pressed to produce a tablet containing 100 mg of the SN triple-bond structured compound.

### Embodiment 4

According to Comparative Example 1, the same method for preparing the pharmaceutical composition containing 100 mg of the active ingredient APSNRI per tablet was employed, the formulation is as follows:
APSNRI 100 mg
sodium ethoxide 5 mg
DMAP 5 mg
polyethylene glycol monoalkyl ether 20 mg
pregelatinized starch 40 mg
microcrystalline silica 10 mg

### Detailed Preparing Method

Tablet preparation: the SN triple-bond structured compound, sodium ethoxide, DMAP, polyethylene ether, pregelatinized starch, and microcrystalline silica, employing direct compression method, were mixed uniformly and pressed to produce a tablet containing 100 mg of the SN triple-bond structured compound.

### Comparative Example 7

For preparing the pharmaceutical composition containing 100 mg of the active ingredient (SN triple-bond structured compound: FPSNRI) per tablet, the formulation is as follows:
FPSNRI 200 mg

### Detailed Preparing Method

Tablet preparation: the SN triple-bond structured compound, employing direct compression method, was mixed uniformly and pressed to produce a tablet containing 100 mg of the SN triple-bond structured compound.

### Embodiment 5

For preparing the pharmaceutical composition containing 100 mg of the active ingredient (SN triple-bond structured compound: FPSNRI) per tablet, the formulation is as follows:
FPSNRI 100 mg
DMAP 10 mg
polyethylene glycol monoalkyl ether 20 mg
microcrystalline cellulose 20 mg
pregelatinized starch 40 mg

### Detailed Preparing Method

Tablet preparation: the SN triple-bond structured compound, DMAP, polyethylene glycol monoalkyl ether, microcrystalline cellulose, and pregelatinized starch, employing direct compression method, were mixed uniformly and pressed to produce a tablet containing 100 mg of the SN triple-bond structured compound.

### Embodiment 6

According to Comparative Example 1, the same method for preparing the pharmaceutical composition containing 100 mg of the active ingredient (SN triple-bond structured compound: FPSNRI) per tablet was employed, the formulation is as follows:
FPSNRI 100 mg
polyethylene glycol monoalkyl ether 20 mg
microcrystalline cellulose 10 mg
pregelatinized starch 10 mg

### Detailed Preparing Method

Tablet preparation: the SN triple-bond structured compound, polyethylene glycol monoalkyl ether, microcrystalline cellulose, and pregelatinized starch, employing direct compression method, were mixed uniformly and pressed to produce a tablet containing 100 mg of the SN triple-bond structured compound.

### Embodiment 7

According to Comparative Example 1, the same method for preparing the pharmaceutical composition containing 100 mg of the active ingredient (SN triple-bond structured compound: FPSNRI) per tablet was employed, the formulation is as follows:
FPSNRI 100 mg
polyethylene glycol monoalkyl ether 20 mg
microcrystalline cellulose 10 mg
pregelatinized starch 10 mg
microcrystalline silica 10 mg

### Detailed Preparing Method

Tablet preparation: the SN triple-bond structured compound, polyethylene glycol monoalkyl ether, microcrystalline cellulose, pregelatinized starch, and microcrystalline silica, employing direct compression method, were mixed uniformly and pressed to produce a tablet containing 100 mg of the SN triple-bond structured compound.

Tests were performed on the above formulations of Embodiments and Comparative Examples: 1) stability is determined by measuring the amount of active compound residue; 2) the deprotonation activity of the active compound can be determined by the degree of reaction with piperidines in different environments, i.e., degree of reactivity over time, a) direct reaction; b) neutral environment; c) acidic environment.

The relevant experimental data are shown in Tables 1 and 2.

Table 1: The tablets of Embodiments and Comparative Examples are placed in 100 ml enclosed containers. The active compound in aqueous solution means that the ratio of water to the active compound by weight is 1:1. The active compound in acetic acid aqueous solution means that the ratio of liquid to the active compound by weight is 1:1 and 1 drop of acetic acid solution is added to make the solution PH 4.8; The active compound in a protein amino acid aqueous solution means that the ratio of liquid to the active compound by weight is 1:1 and 2 eq of alanine is added. The active compound is the SN triple bond compound corresponding to the respective Embodiment and Comparative Example.

TABLE 1
Detection results of stability test

| | | | | | | 24 hours | | |
|---|---|---|---|---|---|---|---|---|
| | Storage at 4 degrees for | | | | | | | Active |
| | 6 months - 24 months | | | | | | Active | compound |
| | | Active | Active | Active | Active | Active | compound | content in |
| | | compound | compound | compound | compound | compound | content in | protein |
| | | content | content | content | content | content in | acetic acid | amino acid |
| | | after 6 | after 12 | after 18 | after 24 | aqueous | aqueous | aqueous |
| | Appear- | months | months | months | months | solution | solution | solution |
| Embodiment | ance | (%) | (%) | (%) | (%) | (%) | (%) | (%) |
| Comparative | pale | 77 | 55 | 33 | 8 | 68 | 1 | 71 |
| Example 1 | yellow | | | | | | | |
| Comparative | pale | 89 | 80 | 73 | 64 | 90 | 88 | 90 |
| Example 2 | yellow | | | | | | | |
| Comparative | pale | 88 | 83 | 79 | 68 | 88 | 86 | 85 |
| Example 3 | yellow | | | | | | | |
| Comparative | pale | 90 | 85 | 82 | 72 | 90 | 87 | 89 |
| Example 4 | yellow | | | | | | | |
| Comparative | pale | 78 | 70 | 65 | 56 | 80 | 76 | 81 |
| Example 5 | yellow | | | | | | | |
| Comparative | pale | 78 | 70 | 66 | 59 | 66 | 4 | 75 |
| Example 6 | yellow | | | | | | | |
| Embodiment 1 | pale | 96 | 92 | 89 | 83 | 95 | 93 | 92 |
| | yellow | | | | | | | |
| Embodiment 2 | pale | 93 | 89 | 85 | 78 | 89 | 87 | 90 |
| | yellow | | | | | | | |
| Embodiment 3 | pale | 90 | 87 | 83 | 80 | 87 | 86 | 89 |
| | yellow | | | | | | | |
| Comparative | pale | 88 | 57 | 63 | 50 | 86 | 78 | 79 |
| Example 7 | yellow | | | | | | | |
| Embodiment 5 | pale | 90 | 85 | 78 | 71 | 89 | 88 | 83 |
| | yellow | | | | | | | |
| Embodiment 6 | pale | 87 | 79 | 69 | 61 | 88 | 85 | 87 |
| | yellow | | | | | | | |

Table 1 shows that the active compound has a good stability. The tests in Table 2 are further performed.

Table 2: a. The reaction in piperidine refers to the reaction between 5 mg of a propoxy-λ⁶-sulfanenitrile and 0.5 ml of piperidine for 24 h in a 40-degree water bath. b. The reaction of piperidine in anhydrous ethanol means that 5 mg of the propoxy-λ⁶-sulfanenitrile is firstly dissolved in about 0.2 ml anhydrous ethanol, and then 0.5 ml piperidine is added and the reaction is carried out for 24 h in a 40-degree water bath. c. The reaction of piperidine in protein amino acid aqueous solution (the pharmaceutical composition is dissolved in anhydrous ethanol) means that 5 mg of the propoxy-λ⁶-sulfanenitrile is first dissolved in about 0.2 ml anhydrous ethanol, then 0.5 ml of piperidine, 3.5 mg of alanine, and 0.5 ml of distilled water are sequentially added, and the reaction is carried out for 24 h in a 40-degree water bath. d. The reaction of piperidine in a weakly acidic protein amino acid aqueous solution (the pharmaceutical composition is dissolved in anhydrous ethanol) means that 5 mg of the propoxy-λ⁶-sulfanenitrile is firstly dissolved in about 0.2 ml anhydrous ethanol, then 0.5 ml of piperidine, 3.5 mg of alanine (equivalent to 2 eq of the active compound), 0.5 ml of distilled water are sequentially added, later, 2 drops of acetic acid solution is added, and the reaction is carried out for 24 h in a 40-degree water bath.

TABLE 2
Detection results of 24 hr piperidine deprotonation activity of the pharmaceutical
compositions containing SN triple-bond structured compound

| | | | | | | | | | d. Reaction of piperidine | | |
|---|---|---|---|---|---|---|---|---|---|---|---|
| | | | | | | c. Reaction of piperidine | | | in weakly acidic protein | | |
| | | | | | | in protein amino acid | | | amino acid aqueous | | |
| | | | | | | aqueous solution (with | | | solution (with the | | |
| | | | b. Reaction of | | | the pharmaceutical | | | pharmaceutical | | |
| | | | piperidine in | | | composition being | | | composition being | | |
| | a. Reaction in | | anhydrous | | | dissolved in anhydrous | | | dissolved in anhydrous | | |
| | piperidine (%) | | ethanol (%) | | | ethanol) (%) | | | ethanol) (%) | | |
| Embodiment | 12 h | 24 h | 12 h | 24 h | 24 h | 12 h | 24 h | 24 h | 12 h | 24 h | 24 h |
| Comparative | 76 | 98 | 0 | 2 | 92 | 0 | 2 | 4 | 1 | 3 | 6 |
| Example 1 | | | | | | | | | | | |
| Comparative | 72 | 87 | 0 | 58 | 85 | 0 | 36 | 76 | 8 | 39 | 72 |
| Example 2 | | | | | | | | | | | |
| Embodiment 1 | 68 | 99 | 8 | 66 | 92 | 3 | 26 | 90 | 8 | 42 | 85 |
| Embodiment 4 | | | | | | | | | | | |
| Comparative | 72 | 99 | 2 | 5 | 91 | 2 | 4 | 58 | 1 | 3 | 46 |
| Example 7 | | | | | | | | | | | |
| Embodiment 5 | 59 | 99 | 48 | 89 | 99 | 39 | 66 | 88 | 8 | 42 | 82 |

Through these tests, it can be concluded that the stability, deprotonation activity, alkylation activity, targeting activity and other utilities in different environments of the SN triple bond compounds are significantly improved when being prepared into the pharmaceutical composition, which lays the foundation for the effective application of such pharmaceutical composition in anti-tumor and other drugs in the future.

When the numerical ranges are given by the embodiments, it is to be understood that the two endpoints of each numerical range and any value between the two may be selected unless otherwise stated. Unless otherwise defined, all technical and scientific terms used in the present disclosure have the same meaning as commonly understood by one skill in the art. In addition to the specific method, equipment, and material used in the embodiments, any method, equipment, and material in the existing technology similar or equivalent to the method, equipment, and material mentioned in the embodiments of the present disclosure may be used to realize the present disclosure according to the knowledge of those skilled in the art and the record of the present disclosure.

1. A pharmaceutical composition, comprising, by mass percent:

| | |
|---|---|
| SN triple-bond structured compound | 2%~98%; |
| stabilizer protectant | 0.5%~97%; |
| pharmaceutical excipient | 0.1%~55%. |

2. The pharmaceutical composition according to claim 1, further comprising a lubricant, wherein the pharmaceutical composition comprises, by mass percent:

| | |
|---|---|
| SN triple-bond structured compound | 40%~85%; |
| stabilizer protectant | 0.5%~8%; |
| pharmaceutical excipient | 5%~55%; |
| lubricant | 2%~18%. |

3. The pharmaceutical composition according to claim 1, wherein the pharmaceutical composition is dissolved in a solvent when using; preferably, the solvent is anhydrous ethanol.

4. The pharmaceutical composition according to claim 1, wherein the SN triple-bond structured compound is selected from APSNRI and FPSNRI.

5. The pharmaceutical composition according to claim 1, wherein the stabilizer protectant is one or more of the following components: sodium alcoholates, alcohols, alcohol polymers, pyridines, and proton-deficient hydrophobic oils.

6. The pharmaceutical composition according to claim 1, wherein the pharmaceutical excipient is one or more of the following components: microcrystalline cellulose, pregelatinized starch, and capsules.

7. The pharmaceutical composition according to claim 2, wherein the lubricant is one or more of the following components: talc powder, magnesium stearate, and microcrystalline silica.

8. A method for preparing the pharmaceutical composition according to claim 1, comprising mixing the SN triple-bond structured compound, the stabilizer protectant, and the pharmaceutical excipient.

9. The method for preparing the pharmaceutical composition according to claim 8, wherein the method further comprises adding a lubricant to a mixture of the SN triple-bond structured compound, the stabilizer protectant, and the pharmaceutical excipient.

10. A Use of the pharmaceutical composition according to claim 1 in drugs for inhibiting cell reproduction and anti-tumor.

11. The pharmaceutical composition according to claim 5, wherein the stabilizer protectant is one or more of the following components: sodium ethoxide, 4-dimethylaminopyridine, polyethylene glycol monoalkyl ether, and anhydrous ethanol.

12. The pharmaceutical composition according to claim 7, wherein the lubricant is microcrystalline silica.
